(19) United States
(12) Patent Application Publication
(10) Pub. No.: US 2008/0118445 A1
(43) Pub. Date: May 22, 2008

(54) Dental Appliance Cleaners and Methods for Making and Using Them

(75) Inventors: Robert K. Larsen, Sandy, UT (US); Calvin D. Ostler, Riverton, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO GROUP, INC., West Jordan, UT (US)

(21) Appl. No.: 12/019,493

(22) Filed: Jan. 24, 2008

Related U.S. Application Data
(63) Continuation-in-part of application No. 10/922,804, filed on Aug. 20, 2004.

(51) Int. Cl. A61K 6/00; A61C 17/00

(52) U.S. Cl. 424/51; 433/216; 134/2; 510/116

(57) ABSTRACT

The addition of the Iodide ion by way of iodide salts, such as sodium iodide and potassium iodide, to a peroxide such as hydrogen peroxide in a basic medium yields free radical oxygen and water; generating large amounts of heat and depleting the Hydrogen Peroxide in a matter of minutes. The free radical oxygen generated in this reaction can be utilized to oxidize organic molecules that produce offending stains on select items, including artificial teeth and other dental appliances. Once the free radical oxygen has oxidized the offending molecule the color is lost and the solubility changes allowing any loose fragments of the offending molecule to be washed away in the solvent. The iodide ion catalyzes the reaction allowing for precise control over the speed at which the stain is removed without the need for other expensive, cumbersome energy adding equipment such as lights, lasers, heat sources, etc.

### CROSS-REFERENCES TO RELATED APPLICATIONS

This patent application is a Continuing-in-Part Application and claims benefit to and the priority of its parent, U.S. Utility patent application Ser. No. 10/922,804 filed on Aug. 20, 2004 which is hereby incorporated by reference in its entirety.

### FIELD OF THE INVENTION

The present invention relates to the field of dental appliance cleaners and more particularly relates to such cleaners utilizing a combination of iodide and hydrogen peroxide in a generally basic medium.

### BACKGROUND OF THE INVENTION

Dental appliance cleaners are known in the prior art. Many of these cleaners utilize a peroxide of some form to bleach stains off of teeth, both natural and artificial. It should be noted that the term “teeth” or “tooth” as used in this specification and the appended claims includes both natural and artificial teeth. This goal is accomplished because peroxides tend to be unstable and have a number of free-radical oxygen atoms which bind with the compounds and elements within staining material, thereby removing the stain's color, and sometimes the staining material itself. Various methods of improving peroxide performance are also known in the prior art, as anything that will stimulate free-radical oxygen production will generally improve basic performance of a bleaching product when such production is controlled as to time, place, and manner. The use of iodine, in particular potassium iodide, with hydrogen peroxide is also known in the field of antiseptics. The use of the hydrogen peroxide as an astringent and the iodine as an antiseptic provide a useful combination when treating minor cut and abrasions. These combinations rely on the disinfecting power of the iodine for their utility, and prefer that the iodine is active and present. This occurs in an acidic environment and leaves the resultant solution of iodine and hydrogen peroxide with the all too familiar reddish-brown staining color associated with antiseptic iodine. There is even some prior art that suggests the use of potassium iodide and peroxide as a cleaner for contact lenses, which requires a pH above 6 in order to limit the iodine coloration, but this still relies on the iodine as a disinfectant and is stated to be used in a preferred pH range of around 7. Starting at a level of approximately 7.5 pH, the relation between tri-iodide molecules and oxygen radicals in the solution changes as the iodine is kept bound in solution as a catalyst and more radicals are released. While it is known that O₂ is formed from the reaction, free-radical Oxygen production from the reaction has been, at best, ignored. The parent Application has gone into extensive detail in the prior art and the reader is directed to that discussion, which has already been incorporated by reference above.

The present invention, in its preferred embodiment, is a dental appliance cleaner presented in a binary solution system, utilizing iodide salts such as potassium iodide and sodium iodide as a catalyst for generation of free-radical oxygen. The cleaner is kept in a binary solution, having two separate components that are combined when desired to be used.

The present invention represents a departure from the prior art in that the appliance cleaner of the present invention utilizes iodide as a catalyst for creating bleaching oxygen radicals in peroxides, thereby increasing whitening effectiveness.

### SUMMARY OF THE INVENTION

In view of the foregoing disadvantages inherent in the known types of dental appliance cleaners, this invention provides an improved dental appliance cleaner with greater efficiency. To accomplish these objectives, the cleaner, in its most basic embodiment, comprises a peroxide based active component and an iodide based catalyst that are kept separate until use.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a depiction of the bleaching, or whitening, reaction.

FIG. 2 depicts a rigid two-chambered canister mixing and delivery system for a two-part bleach or whitener.

FIG. 3 depicts the activation phase of the bleach on pressed tablet and capsule formats.

FIG. 4 depicts the step of measuring the bleach components, stored as depicted in FIG. 2, in a mixing container.

FIG. 5 depicts the steps of soaking a dental appliance in the bleach.

### DETAILED DESCRIPTION OF THE PREFERRED EMBODIMENT

With reference now to the drawings, the preferred embodiment of the bleaching gels are herein described. It should be noted that the articles “a”, “an”, and “the”, as used in this specification, include plural referents unless the content clearly dictates otherwise.

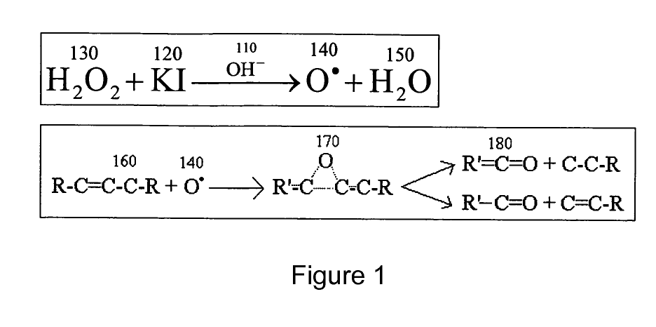

With reference to FIG. 1, it is well established that the free radical oxygen atoms (140) liberated from peroxides such as hydrogen peroxide 130, carbamide peroxide, and salts of peroxides formed from the alkali and alkaline earth metals, readily attack and oxidize organic molecules 160 that comprise the stains in discolored teeth. It is also well established that a release of free radical oxygen atoms from the peroxides can be accelerated by the addition of heat, light and/or chemicals; specifically chemicals that raise the pH of the peroxide environment. A lengthy dissertation of the exact mechanisms is discussed in prior work found in U.S. Pat. No. 6,116,900, “Binary energizer and peroxide delivery system for dental bleaching” which is herein incorporated by reference.

For whitening and bleaching purposes, the addition of the Iodide ion by way of potassium iodide 120, or another iodide salt such a sodium iodide, to a peroxide such as hydrogen peroxide 130 in a basic medium 110 yields free radical oxygen 140 and water 150; generating large amounts of heat and depleting the hydrogen peroxide fairly rapidly given a set relative amount of iodide in the system. The free radical oxygen 140 generated in this reaction can be utilized to oxidize organic molecules that produce offending stains 160 on select items, including teeth. Once the free radical oxygen has oxidized the offending molecule 170 the color is lost. Solubility changes allow any colorless oxidized fragments 180 of the offending molecule to be washed away in the solvent.

It should be noted that earlier uses of iodine and peroxide were antiseptic in nature and utilized free iodine (I₂). The uses of these compositions required the medium to have pH in a neutral to acidic ranges, as free iodine is eliminated as pH is raised to a basic range, beginning somewhere between a pH of 6.5 and 7.5. This invention does not use free iodine. In the basic range, in particular between 7.5 and 8.5, the peroxide decomposition yields a large number of oxygen free radicals in a short period of time while free iodine is eliminated. This combination is especially effective in bleaching applications. As such, any free iodine left in solution is of a negligible amount and not considered relevant to the invention. The reaction levels at a pH of 8.5, when no free iodine is left in the system. Oxygen free radical production flattens at a pH of 8.0. At pH levels above 9.0, the basic nature of the system begins to have a deleterious effect on human tissue, so limiting the system to a maximum pH of 9.0 is desired. As such, any pH range from 7.5 to 9.0 is useful, in particular between 7.5 and 8.5, inclusively.

Ideally, the composition may be thickened into a gel form or it may retain an aqueous form. The term “gel” is defined in this document, as a product that, when applied to the teeth and will tend to adhere to the teeth rather than immediately running off in order to aid in providing a whitening treatment. Therefore the “gel” could also be a thick paste or a very runny or “loose gel.” A gel may be created with or without a thickener or viscosity increaser. The exact formulations for various gels has been exhaustively studied and reported. Any gel that is stable can be utilized. Examples of gelling materials include but are not limited to the long list of polyacrylic acid thickeners most commonly sold under the trade name Carbopol by the BF Goodrich Company; the gum thickeners such as guar gum and xanthane gum; the cellulose thickeners such as methyl cellulose, sodium carboxymethyl cellulose, hydroxyethyl cellulose, hydroxpropyl methyl cellulose and hydroxymethyl propyl cellulose; glycerin and its derivatives; the silica thickeners such as fumed silica and silica aerogel thickener; glycol and its many derivatives such as propylene glycol, polyethylene glycol, and polypropylene glycol; polyoxyethylene polyoxypropylene block copolymeric thickeners available under the trade name PLURONIC available from BASF, cross-linked copolymers of acrylic acid and a hydrophobic comonomer available under the trade name PEMULEN from the BF Goodrich Company, and other thickeners such as sorbitol and polyvinyl alcohol. Polyvinylpyrrolidone is particularly attractive as it provides a gel that is stable across a wide range of pH values. Polyvinylpyrrolidone is also an iodophor. An iodophor is any surface active agent or polymer that acts as carriers and solubilizing agents for iodine. Virtually any thickener may be used provided that it is safe for human exposure and stable in the environments. All of these thickening agents are readily available from the standard chemical sources such as Sigma-Aldrich of Milwaukee, Wis. and Spectrum Chemicals of Gardena Calif. Examples of gels are provided in the parent application referenced above and are specifically incorporated here by reference.

An additional color change feature is enabled first by way of the rapid pH changes involved in the ion catalyzed “decomposition” of peroxide and, second, the highly aggressive oxidation and cleavage of organic molecules by the free radicals produced. For instance if an indicator such as thymolphthalien, which is blue in the very basic range and clear in the near neutral range, was combined with a dye that is readily attacked and destroyed by the free radicals, such as betacarotene, FD&C Red 40, or Amaranth, in the non-peroxide compartment, when the peroxide compartment and the non-peroxide compartment contents are mixed the color would immediately change from indigo blue to red. Over a time frame determined by the concentration of dye present, the red color would fade leaving a clear-colorless solution. If allowed to stand long enough the system, as it rebounds to the basic side, would turn a light blue color. The color could be used to demonstrate the system is active as the indigo blue turns to red. The red color serves as an indication that the peroxide is exhausted as the red color fades. Additionally, FD&C Yellow 5 could be added. Yellow 5 is stable in the presence of free radicals generated by this system. The color would then go from indigo blue, to orange, to yellow to green—depending on the various concentrations. A number of combinations of pH indicators and dyes are possible with the system and could lend themselves to a variety of uses.

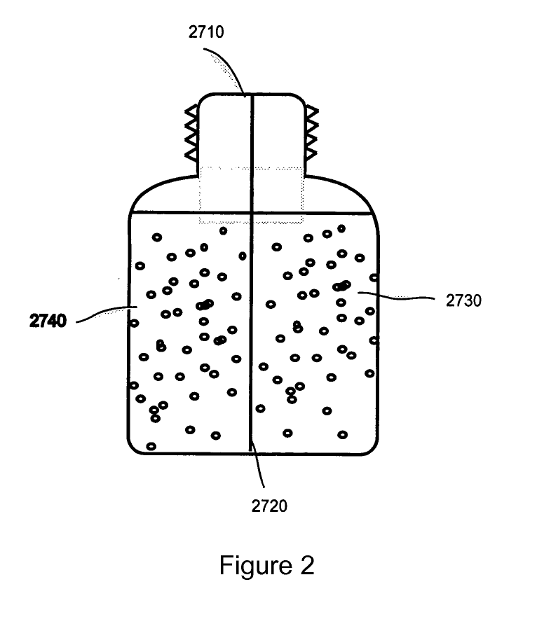

The delivery mechanism and method can be any system that keeps the two components separate until immediately prior to use. Maintaining separation of the two components is necessary as the reaction between the peroxide and iodide will deplete the peroxide too rapidly for the composition to be manufactured, shipped, and stored as a combined solution. Delivery mechanisms can be as simple as two separate containers in which appropriate amounts of each component are removed, placed into a mixing dish, mixed, and then applied to the teeth. For convenience they can include various two component dispensers that automatically dispense appropriate amounts of both components when force is applied such as the double chambered container 2710 as illustrated in FIG. 2. In such a delivery system, a wall 2720 divides container 2710 so that the peroxide containing component is maintained in its own chamber 2730 which is isolated from the non-peroxide containing component which is in its own chamber 2740.

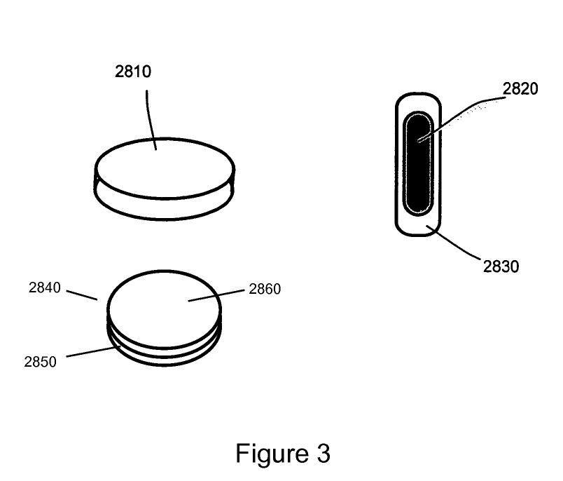

Alternatively, the consumer could apply the mixture themselves by way of the now popular “tablet” method as is illustrated in FIG. 3. The mixture would be pressed into two components, an iodine activation component 2850 and a peroxide base component 2860. The components may be kept separate or may be pressed into a single tablet 2840. The user merely adds the components to water and then adds the appliances. A liquid filled 2820 capsule 2830 may also be utilized. Another strategy would be to provide a single tablet 2810 or capsule comprising the iodine based activation component and add it to consumer grade hydrogen peroxide rather than water.

Many other systems are possible. The above examples are offered for illustrative purposes and are not intended to limit the delivery systems to the offered examples.

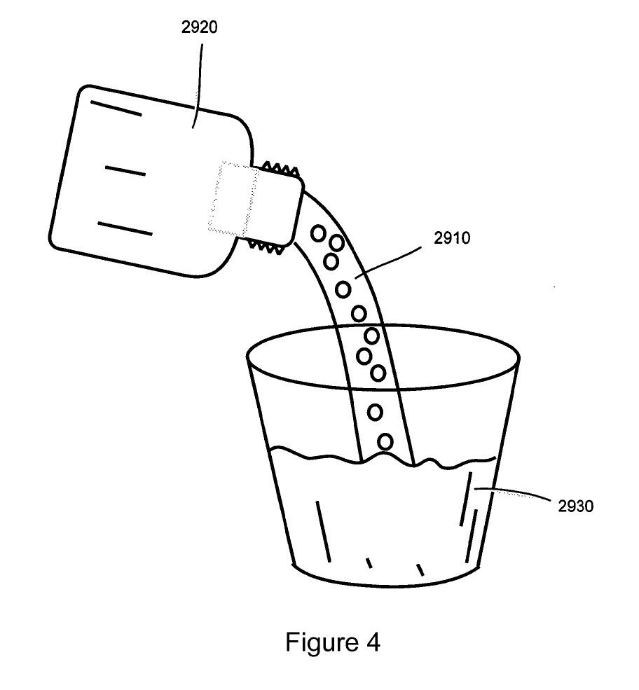

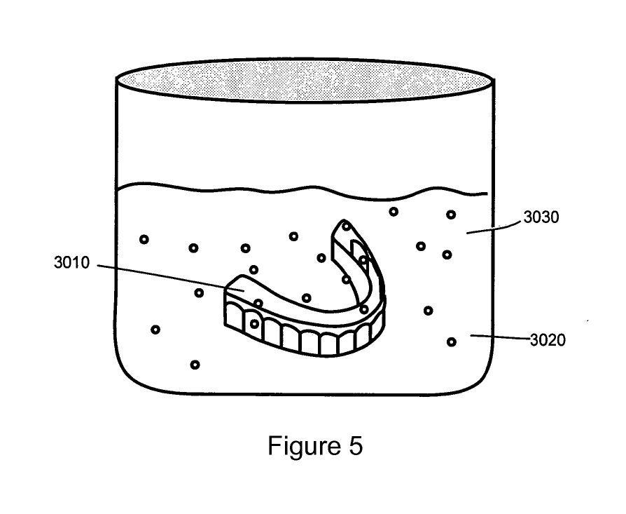

The resultant mixture of the two bleach components into a powerful and effective bleach or whitener can be applied to the dental appliance by the consumer in many different ways. For instance a consumer, refer to FIG. 4, could pour the mixture 2910 from the double chambered container 2920 into a mixing container 2930. The consumer then soaks the appliance 3010 in the bleach 3020 as normal (FIG. 5). If color indicators are used, the user may watch the change in color to know when the cleaner has finished cleaning the appliance (e.g. the liquid becomes clear).

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method of cleaning a dental appliance, the method comprising the steps of:
a. combining an energizer phase containing a compound of iodine with a peroxide phase, forming a bleach with a pH greater than 7.5;
b. applying the bleach to a dental appliance

2. The method as recited in claim 1 wherein said bleach has a pH between 7.5 and 8.5 inclusively.

3. The method as recited in claim 1 wherein said bleach has a pH between but not equal to 7.5 and 8.5.

4. The method as recited in claim 1 wherein said energizer phase includes potassium hydroxide.

5. The method as recited in claim 1 wherein said energizer phase includes both potassium hydroxide and potassium iodide.

6. The method as recited in claim 1 wherein said energizer phase includes a compound of potassium.

7. The method as recited in claim 1 wherein said energizer phase includes a hydroxide and an iodide.

8. The method as recited in claim 1 wherein said energizer phase includes a thickener.

9. The method as recited in claim 1 further comprising a thickener selected from the group consisting of polyvinylpyrrolidone, polyvinyl alcohol and glycerin.

10. The method of claim 1, wherein the dental bleach is in the form of a gel that is applied to the dental appliance.

11. The method of claim 1, the bleach being aqueous and at least one phase is provided initially as a pressed tablet that is dissolved into the bleach.

12. A dental appliance cleaning and bleaching system comprising:
a vessel, further comprising:
an energizer chamber within said vessel,
a oxygen radical chamber within said vessel,
wherein contents of said energizer chamber may be mixed with contents of said oxygen radical chamber to form a useful dental bleach having a pH of at least 7.5,
an energizer phase located within said energizer chamber,
said energizer containing a compound of iodine,
an oxygen radical phase located within said oxygen radical chamber, and
said oxygen radical phase including an oxygen-containing medium; and
a pH sensitive color indicator.

13. A system as recited in claim 12, the bleach having a pH between 7.5 and 8.5 inclusively.

14. A system as recited in claim 12 wherein said phase includes potassium hydroxide.

15. A system as recited in claim 12 wherein said energizer phase includes potassium iodide.

16. A system as recited in claim 12 wherein said energizer phase includes both potassium hydroxide and potassium iodide.

17. A system as recited in claim 12 wherein said energizer phase includes a compound of potassium.

18. A system as recited in claim 12, the bleach having a pH between but not equal to 7.5 and 8.5.

19. A system as recited in claim 12 wherein said energizer phase includes a hydroxide and an iodide.

20. A system as recited in claim 12 wherein mixture of said energizer phase with said oxygen radical phase results in release of oxygen ions that have a beneficial dental appliance whitening effect.
